(19) United States
(12) Patent Application Publication
(10) Pub. No.: US 2008/0114292 A1
(43) Pub. Date: May 15, 2008

(54) Infusion system having an infusion unit and a remote control unit

(76) Inventors: Juergen Rasch-Menges, Schwetzingen (DE); Hans-Peter Haar, Wiesloch (DE); Andreas Poredda, Mannheim (DE); Ulrich Haueter, Grosshochstetten (CH)

Correspondence Address: ROCHE DIAGNOSTICS OPERATIONS INC., 9115 Hague Road, Indianapolis, IN, 46250-0457, US

(21) Appl. No.: 11/694,302

(22) Filed: Mar. 30, 2007

(30) Foreign Application Priority Data
Mar. 30, 2006 (EP) 06 006 679.2

(51) Int. Cl. A61M 31/00

(52) U.S. Cl. 604/65

(57) ABSTRACT

An infusion system comprises an infusion unit for infusing fluid into a user's body and a remote control for wirelessly receiving or sending signals to the infusion unit. The infusion system is capable of operating in two separate modes, namely a remote control mode and a direct control mode. In the remote control mode, an infusion command is generated by an input devise of the remote control. The verification to a user that the command has been received, executed or transmitted is outputted by an output device in the remote controller. In the direct control mode the infusion command is generated by an input device of the infusion unit. The verification to a user that the command has been received, executed or transmitted is outputted by an output device in the infusion unit.

### REFERENCE TO RELATED APPLICATIONS

The present application is based on and claims priority to European Patent Application No. 06008679.2, filed Mar. 30, 2006, which is hereby incorporated by reference in its entirety.

### TECHNICAL FIELD

The present invention generally relates to an infusion system comprises an infusion unit for infusing fluid into a user's body and a remote control for wirelessly receiving or sending signals to the infusion unit. More specifically, the infusion system is capable of operating in two separate modes, namely a remote control mode and a direct control mode.

### BACKGROUND

Such devices are used in diabetes treatment to equalize the insulin balance of the patient by injecting insulin. The equalization of the insulin balance is important because both an insulin dose being too high and also an insulin dose being too low are quite harmful for the patient.

Therefore, multiple devices are known in the prior art, which operate either as “stand-alone devices”, i.e., as individual devices, or additionally have a remote control for controlling the infusion device. Remote control has the advantage that the infusion device can be worn on the body under the clothing and can be operated and controlled comfortably via the remote control.

WO 01/70307 A1 suggests an infusion unit with a shaft which is worn on the body. A “communication key” can be inserted into the shaft to control the infusion unit by a remote control. The remote control can communicate with the infusion unit either via a cable connection or via a wireless connection. In addition to a special remote control device, the possibility of using a computer or a handheld computer for the remote control is also provided for programming the infusion unit. This is preferable, in particular, if more complex programming of the infusion unit is to be performed by medical personnel. The specific remote control is sufficient for simple operation of the infusion unit. Only the bolus rate or the basal rate can be changed and/or set. The communication key can also be removed, however. In this case, the infusion unit operates as an individual device and can not be remote controlled.

A remote controllable infusion apparatus is also known from EP 1109586 B1. In addition to the infusion device, a remote commander (remote control unit) is part of the system described therein. The infusion device can be operated and programmed via the remote control unit. Since maintaining the infusion quantity is very important for the health of the patient, it is suggested that the infusion control commands transmitted to control the infusion unit be acknowledged. For this purpose, verification signals in the form of a visual or acoustic display or a vibration are outputted at the infusion device when a command has been received from the remote control unit. A further verification signal is outputted when the command received from the remote control unit is executed. Thus, the user can, also with remote control of the infusion device, monitor the transmission of the commands to and the execution of the commands by the infusion device via the verification signals. The infusion pump is continuously ready to receive in order to recognize and acknowledge the signals transmitted by the remote control unit.

### SUMMARY

The present invention generally relates to an infusion system having an infusion unit and a remote control unit, the infusion unit being arranged and adapted to be located outside the body and to infuse a liquid into the body. The infusion unit has a housing having a fluid reservoir for receiving the liquid, an input device for inputting infusion control commands, an output device and a communication device for wirelessly transmitting signals to and receiving signals from the remote control unit. The remote control unit has a housing, an input device for inputting infusion control commands, an output device and a communication device for wirelessly transmitting signals to and receiving signals to and from the infusion unit. At least one of the infusion control commands generated by actuating one of the input devices is a command requiring verification, whose transmission and/or execution is verified by a verification signal perceivable by the user, which is outputted by one of the output devices.

One of the objects of the present invention to suggest an infusion system which is improved in its operation, to make the operation more secure and tolerant of errors, in particular, the uncertainty factor of the overall system given by the man-machine interface is to be reduced.

The infusion system according to the present invention comprises having an infusion unit and a remote control unit. The infusion system is adapted for operating in two operating modes. The system includes a trigger device, which switches between the two operating modes, namely a remote control mode and a direct control mode. In the remote control mode, at least one infusion control command, which requires verification, is generated by the input device of the remote control unit and the corresponding verification signal is outputted by the output device of the remote control unit. In the direct control mode, at least one infusion control command, which requires verification, is generated by the input device of the infusion unit and the corresponding verification signal is outputted by the output device of the infusion unit. The system operates either in one or the other mode.

The commands for controlling the infusion, for example, for setting the “basal rate” or for a temporary infusion increase (“bolus”) are referred to as infusion control commands. Those commands are inputted at either the input device of the remote control unit or the infusion unit. Of course, other commands can also be inputted at the input devices, for example, to change a display or to set an internal clock. However, these are not infusion control commands in the meaning of the present invention.

The robustness of the infusion system is enhanced if the verification signal for acknowledging the input of an infusion control command is always (at least also) outputted to the unit of the system at which the command input has occurred. Although the change of the output location of the verification signal first appears to be a reduction in comfort, which apparently results in greater complexity and thus makes it more difficult to assign the verification signals, it was established in the context of the experiments, which the present invention is based on, that a significant increase of the operational reliability is achieved. The user receives a direct confirmation by the “locally generated” verification signal that he has performed an input at the appropriate unit (remote control unit or infusion unit). This information is redundant—from the viewpoint of information theory—because the user himself has inputted the command at the corresponding unit. However, it has been established that this redundancy increases the operational reliability.

It is to be considered that an infusion control command typically comprises a sequence of individual commands which are inputted by pressing keys. It has also been established that the capability of the user to input this sequence correctly and completely at the particular selected input device is significantly improved by the present invention by the locally outputted verification signal. For example, an additional insulin infusion (“bolus”) is programmed by a series of key presses, which are each verified. The local feedback given, if the present invention is used, intuitively ensures that the user also performs the further key presses of a command sequence consequently (at the same input unit).

In addition to this improvement of the operation, the present invention typically also results in savings of the power consumption, because the components not required in the particular operating mode can be turned off. For example, in the direct control mode, the communication device of the infusion unit is turned off, in the remote control mode, the output device of the infusion unit can be disconnected from the power supply. This power savings is important because the operating time at a given battery capacity is thus increased.

The system is configured in such a manner that the switch between the operating modes is not automatic, but rather requires user action. The user can decide whether he wishes to operate the infusion system in the remote control mode or in tie direct control mode. Thus, the user has the freedom of always acting correctly in a situation. This freedom is connected with the security that due to the clear either-or principle, i.e., either remote control mode or direct control mode, his action is intuitively correct. These aspects are also supported in that the system can optimally react to customer requirements due to identical operating sequences both when operating via the remote control unit and also when operating the infusion unit in the direct control mode. Due to, in known systems, operation is possible in parallel both at the remote control unit and also via key presses at the infusion unit, the user is frequently confused. This can result in incorrect inputs and incorrect operations of the infusion system.

In the remote control mode, all infusion control commands requiring verification are generated by the input device of the remote control unit and all corresponding verification signals are outputted by the output device of the remote control unit. No verification signals are outputted at the infusion unit.

In the direct control mode, all commands requiring verification are generated by the input device of the infusion unit and all corresponding verification signals are outputted by the output unit of the infusion unit. The user operates the infusion pump as a standalone device and inputs all commands directly into the infusion unit. Therefore, in the stand alone mode, the remote control unit is not required and it can be turned off. This allows the infusion system to operate with or without the remote control. For example, if the remote control unit has broken down due to a defect or if it was lost and/or forgotten by the patient, the infusion system according to the present invention can still be operated.

The verification signals can be outputted as visual signals, for example, using one or more control lights or by displaying dear text on a display, as acoustic signals, e.g., in the term of tones or as speech output, or as tactile signals, e.g., vibrations. A combination of these signals is also possible. If different signal types are used in a system, the output can depend on a user setting or on the location of the output, i.e., whether the verification signal is outputted at the infusion unit or at the remote control unit.

In yet another object, the verification signal, that can be recognized by the user, is a “command verification signal” indicating that an infusion control command has been received by the infusion unit.

In yet another aspect of the present invention, the verification signal distinguishable by the user is an “execution verification signal” indicating when an injection control command has been executed by the infusion unit. It can further be differentiated whether the execution of the injection control command has just been started or whether the execution has been ended. Both can be indicated in the form of an execution verification signal.

It is also possible to output multiple verification signals one after another. If an execution verification signal is outputted after the output of a command verification signal. This is referred to as a “double indication”, if two execution verification signals are generated after the command verification signal, one to indicate that the execution of the infusion control command has been started, and one to indicate that the execution of the infusion control command has been finished, this is referred to as a “triple, indication”.

In the remote control mode, a double indication is preferably outputted to the remote control unit when a command requiring verification has been generated by the input device of the remote control unit. Optionally, an additional verification signal can be outputted at the infusion unit, in the direct control mode, the same principle can be performed. Here also, a double indication is preferably outputted. In addition, a verification signal can optionally be outputted at the remote control unit. The different output possibilities can be implemented directly by the manufacturer in the infusion system; however, they can also be selected and/or set by the user. In addition to the situational switching of the operating modes by the user, situational determination of the output of the verification signals is thus also possible.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic diagram of an infusion system having an infusion unit and a remote control unit;

FIG. 2 shows a more detailed block diagram of the infusion unit and the remote control unit from FIG. 1:

FIG. 3 shows a block diagram of an alternative remote control unit;

FIG. 4 shows a block diagram to explain the function of a trigger device;

FIG. 5 shows a schematic diagram of an alternative embodiment of the infusion system with an infusion unit and remote control unit.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

### DETAILED DESCRIPTION

The following description of the preferred embodiment is merely exemplary in nature and is in no way intended to limit the invention or its application or uses.

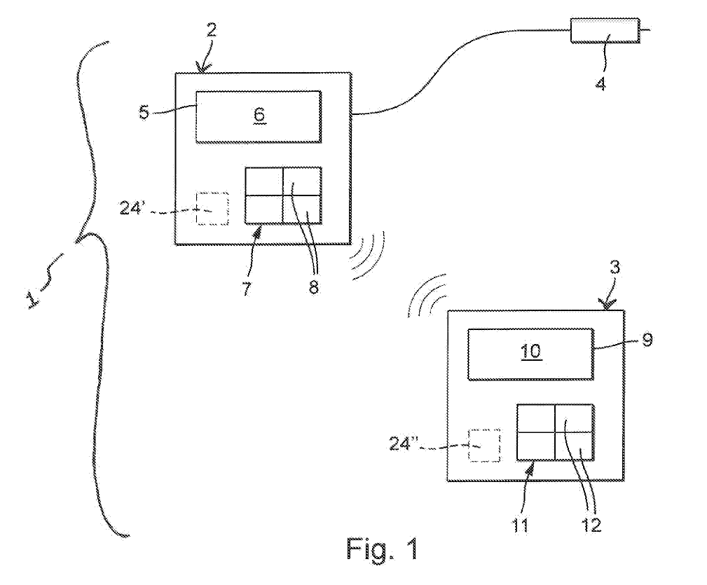

FIG. 1 shows an infusion system 1 having an infusion unit 2 and a remote control unit 3. The infusion unit 2 pumps a liquid, in particular insulin, through thin tubing to an injection unit 4 having a needle. The injection unit 4 is attached to the body of the patient. As shown in a schematic illustration, the infusion unit 2 has an output device 5 implemented as a display 6. Verification signals can be displayed on the display in visual form to acknowledge infusion control commands for controlling the infusion unit 2.

An input unit 7 has keys 8 for controlling the infusion unit 2, The basal rate of the infusion unit 2 can be changed and a bolus can also be set via the keys 8. The complete functional control of the infusion unit 2 can also be performed using the keys 8.

The remote control unit 3 also has an output unit 9, which is implemented as a display 10 for visual display. In addition to the verification signals generated as an answer to the infusion control commands, further indications are also output on the display 10. For example, the time of day or other relevant data can also be displayed.

An input device 11 of the remote control unit 3 substantially corresponds to the input device 7 of the infusion unit 2. The input device 11 is implemented with keys 12. Due to a similar design of the input devices 7, 11 and the output devices 5, 9, the operation at both the remote control unit 3 and also the infusion unit 2 is identical for the user. There is no difference for the patient whether he operates the infusion system 1 via the infusion unit 2 or the remote control unit 3. In this way, operating errors are minimized and the operating comfort is increased.

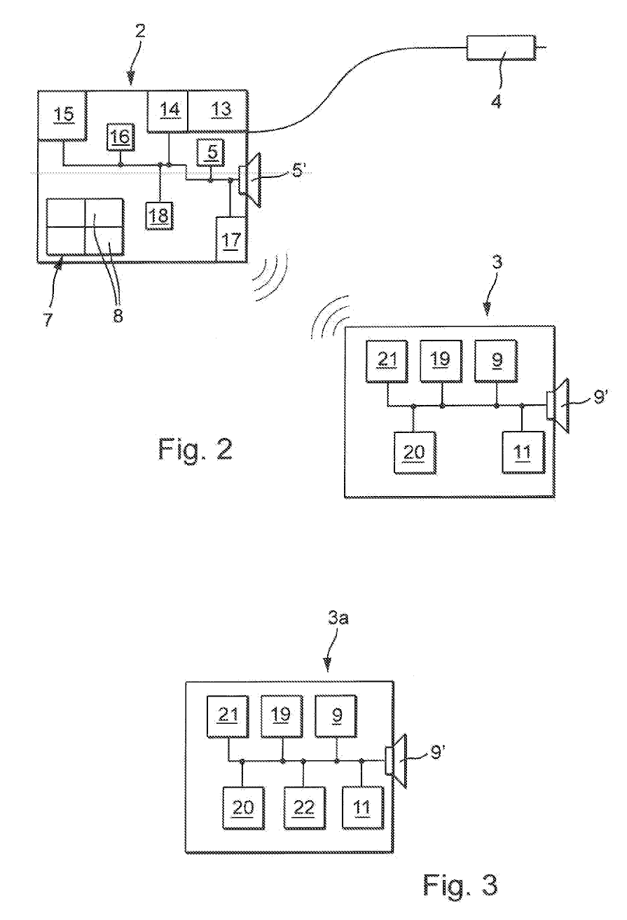

FIG. 2 shows a block diagram of the infusion unit 2 and the remote control unit 3. The infusion unit 2 has a fluid reservoir 13 receiving the liquid, which is applied via the injection unit 4 into the body of the patient. A pump 14 is connected to the fluid reservoir 13. Controlling the pump regulates the infusion quantity to be applied. In particular, the basal rate set in the infusion unit 2 is fixed by the speed of the pump delivery. A bolus optionally to be applied additionally can be controlled by the duration and the amount of a temporary increase of the pump speed.

Furthermore, the infusion unit 2 comprises a power supply unit 15, a microprocessor 16, and a communication device 17 for wirelessly transmitting signals to and receiving signals from the remote control unit 3. The power supply unit 15 supplies all components of the infusion unit 2 with power. The output device 5 is shown as a block. The output device 5 comprises a loudspeaker 5′ that is used for the acoustic output of verification signals at the infusion unit 2. A trigger device 18 is used for switching between the remote control mode and the direct control mode of the infusion system 1.

In addition to the input device 11 and the output device 9, the remote control unit 3 comprises a loudspeaker 9′ for the acoustic output of verification signals. A microprocessor 19 processes the inputs of the input device 11 and the signals transmitted wirelessly from the infusion unit 2, which are received by a communication device 20. The communication device 20 operates bidirectionally. It also transmits signals from the remote control unit 3 to the infusion unit 2. A power supply unit 21 supplies the remote control unit 3 and all components with the required power.

The communication devices 17 and 20 are preferably implemented as transceivers; therefore, they comprise a transmitter for transmitting signals and also a receiver for receiving signals. The transmitted signals are preferably electromagnetic waves. Alternatively, the communication between the communication devices 17 and 20 can be performed via optical signals (in particular via infrared signals). The signals can be coded in the usual ways.

FIG. 3 shows an alternative embodiment of the remote control unit 3a also has a trigger device 22 in addition to the components already described. If a trigger device 22 is provided in the remove control, then, no trigger device 18 may be provided in the infusion unit 2.

In a preferred embodiment of the infusion system 1, the communication device 17 of the infusion unit 2 is deactivated upon the switch into the direct control mode. It is not required in the direct control mode, because no signals have to be transmitted to the remote control unit 3.

The communication device 17 of the infusion unit 2 is especially preferably disconnected from the processor 16 when it is deactivated. Then, the processor 16 transmits no signals to the communication device 17. Additionally or alternatively, the communication device 17 can be disconnected from the power supply unit 15. The deactivation of the communication device 17 is advantageous for multiple reasons. On the one hand, energy is saved, so that the energy stored in the power supply unit 15 lasts longer for operating the infusion unit 2. In addition, interfering influences on the system and the environment are reduced. For example, the shutdown of the communication device 17 can be executed by simple interrupt-controlled routines in the microprocessor 18 monitoring all keys 8 of the input unit. For this purpose, one simple switching transistor per line to be switched can be used.

The output device 5 of the infusion unit 2 is deactivated upon the switch into the remote control mode. Since all verification signals are outputted to the output device 9 of the remote control unit 3 and/or 3a in the remote control mode, the output device 9 does not have to be active. The output device 5 can therefore be shut down. For this purpose, the microprocessor 16 causes a disconnection from the power supply unit 15. When the trigger device 18 or 22 switches the infusion system 1 into the direct control mode, the output device 5 is then reactivated, i.e., supplied with power.

When the microprocessor 18 does not output any signals to the output device 5, the output device 5 can also be deactivated. In addition, the loudspeaker 5′ can also be deactivated. Alternatively, however, it is possible for the loudspeaker 5′ to remain active, so that additional verification signals can be outputted at the infusion unit 2 in the form of acoustic signals. In addition, in spite of the shutdown of the output device 5, a warning tone can be outputted at the loudspeaker 5′ if the liquid quantity in the fluid reservoir 13 falls below a critical value or the voltage outputted by the power supply unit 15 is too low, for example.

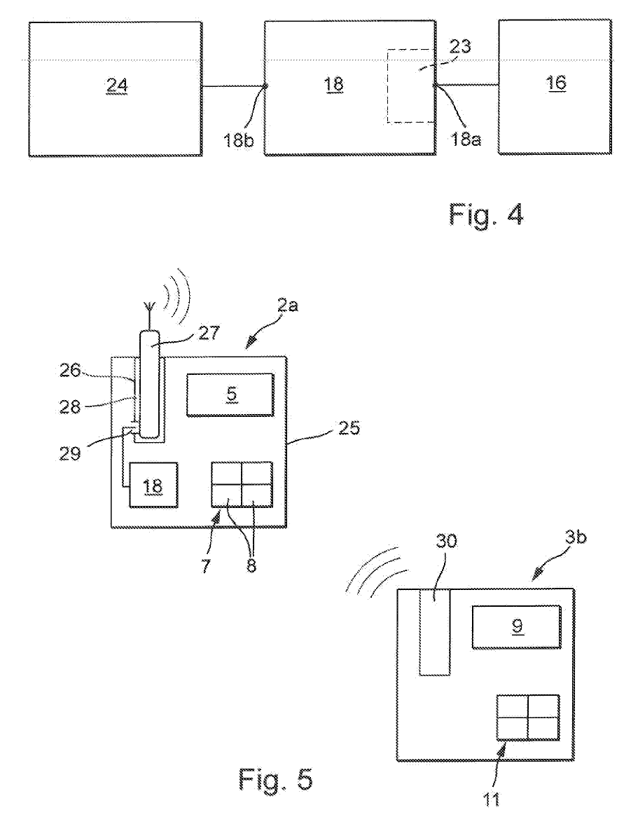

The function of the trigger device 18 is explained on the basis of FIG. 4. In order to cause the switch between the remote control mode and the direct control mode, it generates a control signal for the infusion system at its output 18a, which causes the switch between the operating states using known electronic means. The control signal typically forms a command for the microprocessor 18, which in turn causes the operating mode switch.

The trigger device 18 is in turn controlled by a trigger signal which is transmitted to its input 18b. The means for generating the trigger signal react in some manner to actions of the user or changes in the surroundings of the infusion system. Therefore, they are generally referred to as a trigger sensor 24. Different embodiments of the present invention differ due to the type of the generation of the trigger signal or, in other words, due to different embodiments of the trigger sensor 24.
a) in the simplest case, a manually operable input key specially provided for this purpose (dedicated input key) is used as a sensor for generating the trigger signal. Such an input key may be provided both on the infusion unit 2 and also on the remote control unit 3 (cf. FIGS. 1 and 2; optional input keys 24′ and 24″, respectively). Of course, numerous variations are possible, for example, a pressure sensitive area in a display 6, 10 designed as a “touch screen” (FIG. 1).
b) The sensor 24 can comprise a combination of the keys 8, 12 of the input devices 7, 11, which are provided in any case, with a logic circuit. Then, s combination of key presses (for example, the simultaneous pressing of two specific keys) is interpreted as a manually generated command for switching the operating modes. As a result, a trigger signal is generated and transmitted to the input 18a of the trigger device 18.
c) The sensor 24 can be formed by input change defection electronics. The electronics detect when the user changes the operation from the infusion unit 2 to the remote control unit 3 or vice versa. Therefore, when the system is in the direct control mode and the user inputs an infusion control command at the remote control unit 3, this is recognized by the input change detection electronics and a trigger signal is transmitted to the trigger device 18. All verification signals being used to acknowledge an infusion control command are now outputted to the remote control unit. Thus, such input change detection electronics also form a sensor 24 for generating a trigger signal for the trigger device 18.
d) Finally, the sensor 24 can also be formed by remote control function detection electronics. These recognize when the conditions for the communication between a remote control unit 3 and an infusion unit 2 (both devices turned on, communication devices of both units in operation, secure data transmission ensured) exist, Under these conditions, such remote control function detection electronics generate a trigger signal. The transmission of the trigger signal to the trigger device 18 causes the remote control mode to be turned on.

In cases a) and b), the trigger signal is a manually generated signal.

Case c) is an example of how the trigger signal can be generated as a result of the input of any arbitrary command at one of the input devices 7, 11, preferably as a result of the input of an infusion control command. Such a trigger signal is also referred to as a semi-automatically generated signal.

Case d shows that the trigger signal can also be generated by detecting a signal exchange between the communication devices 17, 20 of the remote control unit 3, 3a, 3b and the infusion unit 2, 2a. A trigger signal of this type is also referred to as an automatically generated signal.

In each of the embodiments described, the switch from a standard mode (such as the direct control mode) into the other mode (remote control mode) can only be triggered by a special trigger signal, while the return into the standard mode is triggered by an (optional) time-dependent control element 23 shown by dashed lines in FIG. 4. The time interval, after which the time-dependent control element 23 causes the switch into the standard mode, can be permanently programmed into the system by the manufacturer or changeable by the user.

The means for implementing the configurations of the sensor 24 described can be implemented very differently by electronics and are known. In particular, the functions implemented in the form of separate electronic components (sensor 24, trigger device 18, and possibly time-dependent control element 23) explained on the basis of FIG. 4 can be implemented partially or completely by software. The above explanations apply in the same manner if the trigger device is located in the remote control unit, of course.

FIG. 5 illustrates a further embodiment of an infusion system 1, comprising an infusion unit 2a and a remote control unit 3b. A recess 26 is provided in the housing 25 of the infusion unit 2a for inserting a communication device implemented as a transceiver module 27. The recess 26 forms a reception chamber 28 fitting to the transceiver module 27. A contact 29 is located in the lower area of the reception chamber 28 for detecting the presence or absence of the transceiver. As soon as the removable transceiver module 27 is inserted into the infusion unit 2a, the contact 29 is closed. Thus, the contact 29 forms a sensor generating a trigger signal. Said trigger signal is applied to the input of the trigger device 18, so that the operating mode is switched into the remote control mode.

The removal of the transceiver module 27 from the reception chamber 28 opens the contact 20. The trigger device 18 switches into the direct control mode. Then, communication with the remote control unit 3b is no longer possible. All inputs are expected at the input device 7 of the infusion unit 2a. Therefore, the trigger signal is manually generated by plugging in and/or removing the transceiver module 27.

During the direct control mode of the infusion system 1, the transceiver module 27 can be stored in a corresponding recess 30 in the remote control unit 3b. In this way, it can not be lost. The transceiver module 27 is only required when the patient wishes to operate his infusion system 1 via the remote control unit 3b and therefore the system must be switched into the remote control mode. The patient then has both the remote control unit 3b and also the transceiver module 27 directly together on hand. He only still has to plug the remote control unit 3b into the infusion unit 2a.

Instead of the transceiver module 27, a removable antenna module can also be provided. The antenna module is used in the same manner as the transceiver module 27.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it wilt be apparent that modification and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. An infusion system comprising:
an infusion unit, wherein the infusion unit is adapted to be located outside the body of a user and to infuse a liquid into the body of the user;
a remote control unit for wirelessly transmitting signals to and receiving signals from the infusion unit; and
a trigger device on the infusion unit or the remote control unit for operating the infusion system in a remote control mode or a direct control mode.

2. The infusion system of claim 1, wherein the infusion unit comprises an input device for inputting at least one infusion control command, an output device for outputting a verification signal perceivable to the user that the at least one infusion control command has been received or executed by the infusion unit, such that in the direct control mode, the at least one command is generated by the input device of the infusion unit and the corresponding verification signal is outputted by the output device of the infusion unit.

3. The infusion system according to claim 2, wherein the output device of the infusion unit is deactivated upon the switching into the remote control mode.

4. The infusion system of claim 1, wherein the remote control unit comprises an input device for inputting at least one infusion control command, an output device for outputting a verification signal that the at least one infusion control command has been received or executed by the infusion unit, such that in the remote control mode, the at least one command is generated by the input device of the remote control unit and the corresponding verification signal is outputted by the output device of the remote control unit.

5. The infusion system according to claim 1, wherein the trigger device switches between the operating modes when a trigger signal is transmitted to its input.

6 The infusion system according to claim 5, wherein the trigger signal is a manually generated signal.

7. The infusion system according to claim 5, wherein the trigger signal is generated as a result of an input of the at least one infusion control command at one of the input devices.

8. The infusion system according to claim 5, wherein the trigger signal is generated by detecting a signal exchange between the remote control unit and the infusion unit.

9. The infusion system according to claim 1, wherein wireless communication capabilities of the infusion unit is deactivated upon the changeover into the direct control mode.

10. The infusion system according to claim 1, wherein the trigger device comprises a time-dependent control element, so that after switching over into the direct control mode, the system is switched back automatically into the remote control mode after a predetermined time.

11. An infusion system comprising:
an infusion unit, wherein the infusion unit is adapted to be located outside a body of a user and to infuse a liquid into the body of the user;
a remote control unit for wirelessly transmitting signals to and receiving signals from the infusion unit;
wherein the infusion unit is capable of operating only in a remote control mode or a direct control mode, such that when at least one infusion control command is inputted to an input device of the infusion unit or the remote control unit, the at least one infusion control command generates a verification signal perceivable to the user that the at least one infusion control command has been received or executed by the infusion unit, the verification signal being outputted to an output device of the infusion unit or the remote control unit;
wherein in the remote control mode the at least one infusion control command is generated by the input device of the remote control unit and the corresponding: verification signal is outputted by the output device of the remote control unit; and
wherein in the direct control mode the at least one infusion control command is generated by the input device of the infusion unit and the corresponding verification signal is outputted by the output device of the infusion unit.

12. The infusion system according to claim 11, wherein the output device of the infusion unit is deactivated upon the switching into the remote control mode.

13. The Infusion system according to claim 11, wherein wireless communication capabilities of the infusion unit is deactivated upon the changeover into the direct control mode.

14. The infusion system of claim 11, further comprising a trigger device for switching between the remote control mode and the direct control mode.

15. The infusion system according to claim 14, wherein the trigger device switches between the operating modes when a trigger signal is transmitted to its input.

16. The infusion system according to claim 15, wherein the trigger signal is a manually generated signal.

17. The infusion system according to claim 15, wherein the trigger signal is generated as a result of an input of the at least one infusion control command at one of the input devices.

18. The infusion system according to claim 15, wherein the trigger signal is generated by defecting a signal exchange between the remote control unit and the infusion unit.

19. The infusion system according to claim 14, wherein the trigger device comprises a time-dependent control element, so that after switching over into the direct control mode, the system is switched back automatically into the remote control mode after a predetermined time.

20. An infusion system comprising:
an infusion unit, wherein the infusion unit is adapted to be located outside the body of a user and to infuse a liquid into the body of the user, the infusion unit comprising:
a housing with a fluid reservoir for the liquid,
an input device for inputting infusion control commands,
an output device, and
a communication device for wirelessly transmitting signals to and receiving signals from the remote control unit;
a remote control unit for communicating with the infusion unit, the remote control unit comprising:
a housing,
an input device for inputting infusion control commands,
an output device,
a communication device for wirelessly transmitting signals to and receiving signals from the infusion unit,
wherein the infusion unit is capable of operating only in a remote control mode or a direct control mode, such that when at least one infusion control command is inputted to the input device of the infusion unit or the remote control unit, the at least one infusion control command generates a verification signal perceivable to the user that the at least one infusion control command has been received or executed by the infusion unit, the verification signal being outputted to the output device of the infusion unit or the remote control unit.

21. The infusion system of claim 20, wherein in the remote control mode the at least one infusion control command is generated by the input device of the remote control unit and the corresponding verification signal is outputted by the output device of the remote control unit.

22. The infusion system of claim 20, wherein in the direct control mode the at least one infusion control command is generated by the input device of the infusion unit and the corresponding verification signal is outputted by the output device of the infusion unit.

23. The infusion system of claim 20, wherein the communication device of the infusion unit is deactivated upon the changeover into the direct control mode.

24. The infusion system of claim 23, wherein the communication device of the infusion unit is disconnected from a processor of the infusion unit and/or a power supply unit of the infusion unit when the communication device is deactivated.

25. The infusion system of claim 20, wherein the output device of the infusion unit is deactivated upon the switching into the remote control mode.

26. The infusion system of claim 20, further comprising a trigger device for switching between the remote control mode and the direct control mode.

27. The infusion system according to claim 26, wherein the trigger device switches between the operating modes when a trigger signal is transmitted to its input.

28. The infusion system according to claim 27, wherein the trigger signal is a manually generated signal.

29. The infusion system according to claim 27, wherein the trigger signal is generated as a result of an input of the at least one infusion control command at one of the input devices.

30. The infusion system according to claim 27, wherein the trigger signal is generated by defecting a signal exchange between the remote control unit and the infusion unit.

31. The infusion system according to claim 26, wherein the trigger device comprises a time-dependent control element, so that after switching over into the direct control mode, the system is switched back automatically into the remote control mode after a predetermined time.
